(19) United States
(12) Patent Application Publication
(10) Pub. No.: US 2004/0101920 A1
(43) Pub. Date: May 27, 2004

(54) Modification assisted profiling (MAP) methodology

(76) Inventors: Czeslaw Radziejewski, Somers, NY (US); Ergang Shi, Ossing, NY (US)

Correspondence Address: REGENERON PHARMACEUTICALS, INC, 777 OLD SAW MILL RIVER ROAD, TARRYTOWN, NY, 10591, US

(21) Appl. No.: 10/699,361

(22) Filed: Oct. 31, 2003

Related U.S. Application Data
(60) Provisional application No. 60/423,017, filed on Nov. 1, 2002.

(51) Int. Cl. G01N 33/53; G01N 33/537; G01N 33/543

(52) U.S. Cl. 435/7.92

(57) ABSTRACT

Methods of utilizing a biosensor platform for the purpose of studying macromolecule interactions is provided. Also provided are methods of sorting monoclonal antibodies directed against a particular antigen into functional groups wherein each group exhibits a characteristic binding profile to the antigen.

### CROSS-REFERENCE TO RELATED APPLICATIONS

This application claims the benefit of U.S. Provisional Application No. 60/423,017, filed 1 Nov. 2002, which application is herein specifically incorporated by reference in its entirety.

### FIELD OF THE INVENTION

This invention generally relates to methods for evaluating macromolecular interactions. In particular, it relates to evaluating antibody/antigen interactions and using the information derived from such evaluation to sort the antibodies into functional groups which can be used as a guide for clone selection, epitope mapping and functional prediction.

### DESCRIPTION OF RELATED ART

Steinrücke et al. (2000) Analytical Biochemistry 286:26-34, describes affinity-tagged helical proteins with unique protease cleavage sites that serve as uniform substrates for in vitro detection of IgA endoprotease. The proteolytic action can be monitored in real time using surface plasmon resonance spectroscopy (Haggarty et al. (2003) J. Am. Chem. Soc. 125:10543-10545) describe a chemical genomic profiling method where the response of genetically similar but not identical cells to pairwise combinations of biologically active small molecules yields a network of chemical genetic interactions.

### BRIEF SUMMARY OF THE INVENTION

The present invention provides a method for evaluating macromolecular interactions utilizing a biosensor platform. In a particular application, the invention provides a method for screening large numbers of monoclonal antibodies (mAbs) directed against a single antigen, and the subsequent sorting of such antibodies into functional groups whose members exhibit a unique yet highly similar binding profile to a modified antigen. This method, termed Modification-Assisted Profiling (MAP), is based in part on an epitope principle which provides that, if a series of independent stable changes are introduced into a macromolecule M, the degree of similarities between the response profiles (patterns) of any two of the mAbs against M reflects the degree of the similarities of these two mAb epitopic locations on macromolecule M. MAP enables one to obtain a nearly complete set of non-redundant monoclonal antibody-producing hybridoma clones and to focus on a small number of hybridoma cultures for further characterization and functional analysis. Thus, it is possible to rapidly identify rare hybridoma clones that produce mAbs having the desired characteristics.

Accordingly, a first aspect of the invention is method of identifying a site of interaction between a first and second macromolecule, comprising the steps of (a) immobilizing the first macromolecule onto at least two biosensor surfaces; (b) treating each biosensor surface containing the immobilized first macromolecule with a different agent, wherein each agent is capable of altering the structure of the immobilized first macromolecule; (c) exposing each treated biosensor surface to the second macromolecule; d) determining an interaction profile of the second macromolecule to the immobilized treated first macromolecule; and (e) identifying a site of interaction between the first and second macromolecules. Such determinations are based on polypeptide sequence information, knowledge of the relationship between a particular chemical or enzymatic modification and the affected amino acid residue(s), as well as the MAP profile.

In specific embodiments, (i) the first macromolecule is a protein and the second macromolecule is a protein that is different from the first macromolecule protein, or a carbohydrate or a nucleic acid; or (ii) the first macromolecule is a carbohydrate, or a nucleic acid, and the second macromolecule is a protein; (iii) the first macromolecule is a ligand and the second macromolecule is a receptor; and (iv) the first macromolecule is a receptor and the second macromolecule is a ligand. In specific embodiments, the ligand is a carbohydrate, nucleic acid, small molecule, protein, or lipid. In still further specific embodiments, the nucleic acid is DNA or RNA, and the protein is a transcription factor.

In a second aspect, the invention features a method of sorting antigen-specific antibodies (mABs) into functional groups, i.e. monoclonal antibodies that share the same or nearly the same epitope, comprising (a) immobilizing the antigen onto at least two biosensor surfaces; (b) treating each biosensor surface with a different agent capable of altering the structure of the immobilized antigen in a specific and stable manner; (c) exposing each treated biosensor surface to the antigen-specific mABs; (d) determining a binding profile of the mAbs to each treated biosensor surface; and (e) sorting the mAbs into functional groups based on the binding profile of the monoclonal antibodies to each treated biosensor surface, wherein mAbs that exhibit similar binding profiles to each treated biosensor surface are sorted into the same functional group, i.e. they have the same or nearly the same epitope.

In a third aspect, the invention a method of sorting unique antigen-specific monoclonal antibodies that mimic a pre-determined function toward the antigen into functional groups, comprising (a) immobilizing the antigen onto at least two biosensor surfaces; (b) treating each biosensor surface with a different agent capable of altering the structure of the immobilized antigen; (c) exposing each treated biosensor surface to the antigen-specific mAbs and a supervising binder, wherein the supervising binder is a different mAb with a known biological function, (i.e. acts as an agonist or antagonist toward certain specific functional aspect of the antigen molecule) or a natural binding partner (ligand) to the antigen (receptor); (d) determining the binding profile of the mAbs and the supervising binder to each treated biosensor surface; and (e) performing an alignment analysis to determine which mAb(s) are most similar to the supervisor based on the binding profile of the monoclonal antibodies and the supervisor binder to each treated biosensor surface.

In specific embodiments, the agents capable of altering the structure of the immobilized antigen or first macromolecule are enzymes. In more specific embodiments, the enzymes are proteolytic enzymes. In particular, specific embodiments, the proteolytic enzymes are trypsin, endoproteinase Glu-C, endoproteinase Asp-N, chymotrypsin, endoproteinase Lys-C, or endoproteinase Arg-C. In other particular embodiments, the enzymes are carbohydrate degrading ezymes such as exoglycosidases (EndoH, O-Glycosidase, and PNGaseF) and endoglycosidases (NANaseI, GALaseI, II, III, IV; HEXase I, II, III, VI; and MANase II). In other embodiments, the enzymes are lipases or endonucleases. Skilled artisans will recognize that many other enzymes may be used in practicing the methods of the invention, with the choice of enzyme being dependent on the nature of the immobilized antigen or first macromolecule (i.e. protein, carbohydrate, lipid, nucleic acid, etc.).

In still other embodiments, the agents capable of altering the structure of the immobilized antigen or first macromolecule are chemical agents. In more specific embodiments, the chemical agents are succinimidyl esters and their derivatives, primary amine-containing compounds, hydrazines and carbohydrazines, free amino acids, homo- and hetero-oligopeptides containing two to twenty residues in length, Tris (2-carboxyethyl) phosphine hydrochloride (TCEP•HCl), N-ethyl-N′-(dimethylamino-propyl) carbodiimide (EDC), iodoacetamide, p-hydroxyphenylglyoxal (HPG), hydrogen peroxide, N-bromosuccinimide, N-acetylimidazole, tetranitromethane, arsanilic acid, dansyl chloride, glutaraldehyde, ninhydrin, or diethylpyrocarbonate (DEPC). Other suitable chemical agents include any primary amine compound, organic compounds that will react with amino acid residue side groups, poly-amino acids, and organic compounds that will react with lipids, carbohydrates, or nucleic acids such as lipid modifying agents selected from the group consisting of reactive compounds that modify lipids by N-ethyl-N′-(dimethylaminopropyl) carbodiimide (EDC)-mediated chemistry; carbohydrate-modifying agents selected from the group consisting of primary amine-containing compounds that modify carbohydrates by periodate-mediated chemistry; and nucleic acid-modifying agents such as methylating agents. Once again, skilled artisans will recognize that many other chemical agents may be used in practicing the methods of the invention depending on the nature of the immobilized first macromolecule.

In a preferred embodiment of the invention, the biosensor platform utilized is a Biacore® biosensor. Other suitable biosensors include IAsys® instruments by Affinity Sensors, a SPR670 by Nippon Laser Electronics, a Bio-Suplar II by Analytical μ-Systems or a Spreeta™ by Texas Instruments. Skilled artisans will recognize that other biosensors can also be used in practicing the methods of the invention.

Other objects and advantages will become apparent from a review of the ensuing detailed description.

### BRIEF DESCRIPTION OF THE FIGURES

FIGS. 1A-1C: Representative Biacore® sensorgrams of modified antigen surfaces.

FIGS. 2A-2B: Normalized response profiles of anti-human Tie2 monoclonal antibodies or angiopoietins to nine modified hTie2-Fc biosensor surfaces.

FIGS. 3A-3C: Pair-wise binding of anti-hTie2 monoclonal antibodies to hTie2 antigen within or among functional groups using standard Biacore® methodology.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only by the appended claims.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are specifically incorporated by reference in their entirety.

General Description

The invention provides a method for evaluating macromolecular interactions utilizing a biosensor platform. Such macromolecular interactions include, but are not limited to, protein/protein, carbohydrate/carbohydrate, lipid/lipid, nucleic acid/nucleic acid, protein/carbohydrate, protein/lipid, protein/nucleic acid, carbohydrate/lipid, carbohydrate/nucleic acid, and nucleic acid/lipid interactions. Skilled artisans will recognize that any interaction between macromolecules is amenable to analysis by the methods of the invention.

In a particular and specific application, the invention provides a method for evaluating the interactions between mAbs and the antigens to which they are directed, enabling a rapid method for sorting the mAbs into functional groups (also called clusters or bins) whose members, called siblings, exhibit a unique and similar binding profile to chemically or enzymatically modified antigen. This is accomplished by any of the methods of: 1) visually examining and grouping, treating each antibody binding response profile exhibited as a graduated bar (as percentage of the control from each modified antigen surface); 2) calculating the determinant value of each antibody binding matrix and sorting all the calculated determinants into groups (see “Calculus—One and Several Variables” 6^{th} Edition by Salas and Einar, pp 715-717, 1990); 3) applying pattern recognition algorithms and related bioinformatic software to the binding response data generated by MAP and classifying the antibodies into functional groups.

Definitions

By the term “biosensor” or “biosensor platform” is meant an analytical device, typically surface plasmon resonance (SPR) detection devices such as Biacore instruments, through which the first molecular coupling, molecular modifications, and the second molecular interaction with the first molecule and its detection are conducted. Such analytical devices can also be microarray devices in which the first molecule and its various modified versions can be dotted or stamped onto a glass surface(s) followed by binding of the second molecule and the subsequent detection of the bound level of the second molecule to each first molecular dot through a typical microarray detection device. Such analytical devices can also be a dot-blotting or western-blotting devices used for proteins or other macromolecular detection where the first molecule and its various modified versions can be dotted onto a sheet surface(s) followed by binding of the second molecule and the subsequent detection of the bound level of the second molecule to each first molecular dot through a typical dot-blot or western-blot detection assay.

“Biosensor surface” means physical flat surfaces, typically gold-coated glass, wherein the gold surface is chemically derivatized for molecular coupling. A non-limiting example is that found with SPR detection devices such as Biacore instruments. The biosensor surfaces can also be extended to a glass surface such as that used in microarray devices. The biosensor surfaces can also be extended to a sheet surface such as polyvinyldifluoride (PVDF) typically used for proteins or other macromolecular detection with a typical dot-blot or western-blot detection assay.

The term “epitope” as used herein means a set of atoms or groups of atoms from an antigen molecule that is recognized by an antibody molecule. This set of atoms or groups of atoms form a specific, non-covalent interacting pocket for a matching set of atoms or groups of atoms, called a “paratope”, from an antibody.

The term “chemically modified” as used herein means the structural changes a macromolecule, for example a protein or polypeptide, undergoes following exposure to a chemical agent. Such structural changes include, but are not limited to, modifying primary amine group typically from the ω-amine of lysine residue by succinimidyl esters, or modifying carboxylic acid groups from aspartic or glutamic acid residues with primary amine-containing compounds to form amide bond typically through a carbodiimide-mediated reaction. Other examples of chemical modifications include those that are typically used for modifying proteins or polypeptides with varying degrees of specificity such as modifying tryptophan residues with N-bromosuccinimide (NBS), modifying tyrosine residues with N-acetylimidazole or tetranitromethane, modifying arginine residues with p-Hydroxyphenyl glyoxal (HPG), modifying histidine residues with iodoacetate, and modifying methionine residues with hydrogen peroxide or N-chlorosuccinimide.

The term “interaction profile” or “binding profile” as used herein refers to a set of pre-arranged normalized binding signals (intensities) of a binder (such as a mAb) to a series of structurally related molecules that the binder binds (such as the antigen molecule that a mAb is directed against).

By the term “functional group” or “cluster” or “bin” as used herein is meant a collection of one or more binders such as mAb that share same or similar binding profiles as measured by the MAP procedure. It is common for members within such “functional group” or “cluster” or “bin” to bind to the same or nearly the same epitope on the antigen. By the term “sibling” is meant a collection of mAbs that either share an identical gene sequence as measured by RT-PCR (Reverse Transcriptase-Polymerase Chain Reaction), these being genetic siblings; or a collection of mAbs that share the same or nearly the same epitope even though their gene sequences are not identical, called functional siblings.

Biosensor Platforms

Affinity-based biosensors employ biological molecules, such as antibodies, receptors, ligands, enzymes, carbohydrates, or nucleic acids, as signal transducers at the interface between solid-state electronics and solution-phase biology. The inherent recognition properties of these biomolecular interactions can be observed and measured by biosensors with a high degree of sensitivity and selectivity (for review, see Baird and Myszka (2001) J. Molecular Recognition, 14:261-268).

Two key advantages of biosensors include the ability to collect data in real-time, thus rapidly providing detailed information about a binding reaction, and second, the binding reaction between interacting biomolecules does not require labeling of the biomolecules, for example, with fluorescent or radioactive labels in order for the binding reaction to be observed. The most established biosensor instruments and technology is currently provided by Biacore AB (Uppsala, Sweden). The Biacore instruments (models 1000, 2000, and 3000) are fully automated, sensor chip-based SPR devices that can accept samples directly from 96-well plates. When docked into one of these instruments, a sensor surface, called a chip, is divided into four independent flow cells that can be operated individually or in a series. This flow-cell configuration allows buffer to pass continuously over the sensor surface, thereby alleviating the need for time-consuming washing steps when exchanging analyte solution for buffer. In addition, continuous flow systems ensure that the ligand is exposed to a constant analyte concentration for the duration of the binding measurement process. Furthermore, the availability of four flow-cells on each sensor chip permits the user to immobilize three different samples and maintain a reference surface within the same sensor chip. The Biacore 2000 and 3000 models are capable of monitoring binding interactions within all four flow-cells simultaneously. The delivery of analyte to each surface in series allows in-line reference subtraction and improved data quality (Myszka (1999) J. Mol. Recogn. 12:279-284; Rich et al. (2000) Curr. Opin. Biotechnol. 11:54-71). Other biosensors such as IAsys® instruments by Affinity Sensors, SPR670 by Nippon Laser Electronics, Bio-Suplar II by Analytical μ-Systems, and Spreeta™ by Texas Instruments can also be used in practicing the methods of the invention.

Chemical Modification of Macromolecule

Modification or alteration of macromolecule (i.e. antigen) structure is effected by either chemical treatment that tends to specifically modify side chains of particular amino acid residues of the antigen protein, or by enzymatic treatment. Typically, nine different types of macromolecular modifications are performed. However, other types and numbers of macromolecular modifications are possible. Non-limiting examples of chemicals that are suitable to effect the chemical alteration or modification include succinimidyl esters and their derivatives, primary amine-containing compounds, hydrazines and carbohydrazines, free amino acids, homo- and hetero-oligopeptides containing two to twenty residues in length, Tris (2-carboxyethyl) phosphine hydrochloride (TCEP•HCl), N-ethyl-N′-(dimethylaminopropyl) carbodiimide (EDC), iodoacetamide and hydrazine, p-hydroxyphenylglyoxal (HPG), hydrogen peroxide, N-bromosuccinimide, N-acetylimidazole, tetranitromethane, arsanilic acid, dansyl chloride, glutaraldehyde, ninhydrin, or diethylpyrocarbonate (DEPC). Skilled artisans will recognize that still many other chemicals could be used in practicing the method of the invention.

Enzymatic Modification of Macromolecule

Non-limiting examples of enzymes, specifically proteases, that are suitable to effect the enzymatic alteration or modification include modified porcine trypsin, endoproteinase Glu-C, endoproteinase Asp-N, chymotrypsin, endoproteinase Lys-C, and endoproteinase Arg-C. Once again, the skilled artisan will readily recognize that other proteases could be used in practicing the method of the invention.

All modifications, are carried out on the macromolecule which is immobilized on a sensor surface. Binding is measured as resonance units (RU) using experimental settings that allow for simultaneously measuring the second macromolecules binding/interaction to all four immobilized macromolecular surfaces including one non-modified and three modified surfaces of each sensor chip. Normalized responses are calculated as percentages of binding responses from each of the three modified surfaces to the control (unmodified) sensor surface. Therefore, the nine response data (%) of each sample are collected by running each sample over three separately prepared sensor chips, each containing a non-modified surface and three differently modified surfaces.

Analysis of Data

The normalized response profiles for each macromolecular interaction is organized into groups using appropriate statistical software. The grouping can also be achieved by calculating the determinant of each response matrix followed by sorting determinants into groups and possibly visually inspecting the gradated color bar column (profile) of each group to verify the grouping results. The entire “grouping process” can be achieved by bioinformatic pattern recognition or data mining computation software. Non-limiting examples of such software include the commercially available programs routinely used by DNA microarray analyses like J-express (DeNova, Inc. Vancouver, British Columbia), Stanford Gene Cluster Software (Stanford University, Calif.), StatSoft of Statistica, or other suitable non-commercial programs developed by skilled artisans.

Method Applications

The methods described herein may be used to explore many macromolecular interactions; for example, identifying and eliminating redundant clones in the hybridoma cloning process. An ideal set of hybridoma clones should be a complete, non-redundant set of clones that encompass all possible linear and non-linear epitopes of the antigen. Such a set will most likely represent every possible structural and chemical feature of the antigen, including unknown structural features. MAP allows the user to obtain information on a large group of hybridoma clones based on their antigen binding profile and eliminates redundant clones from further analysis.

Another application example is in identifying and eliminating redundant siblings from a recombinant antibody sub-library or single chain fragment of variable regions of antibody (ScFv) library. To accomplish this, individual genes for each antibody belonging to a group of related antibodies are genetically engineered using standard molecular biology techniques familiar in the art into expression hosts such as, for example, bacterial cells, CHO (Chinese Hamster Ovary) cells, or into a phage display system. As described herein MAP can be directly applied and can help identify all siblings regardless of their origin and nature.

Yet another application is in identifying desirable hybridoma clones using natural binders. Some of the most useful and most desirable features of antibodies include sensitivity and specificity for detecting antigen molecules in various systems such as stained and/or fixed tissue slices or immunoprecipitation of the antigen from complex mixtures; the ability to mimic the natural ligand or other natural binding partner to the antigen which can make antibodies useful as agonists; and the ability. to prevent the interaction between the natural ligand or other natural binding partner and the antigen which can make antibodies useful as antagonists. Often it is difficult to incorporate the necessary assays into the primary hybridoma screening process to identify antibodies with either agonistic or antagonistic properties. MAP can generate information that reflects the structural relationship between each of the mAbs and their antigen. Therefore, by simply adding the natural ligand or other binding partner molecules (as separate samples) into the screening assay process, and then comparing the response profile similarities among the hybridoma samples with those of the natural binders, the user is able to predict which antibody sibling groups are the best prospects as agonists or antagonists.

Yet another application is for detection antibodies may be discovered by re-screening the complete, non-redundant monoclonal antibody set defined by the MAP using various immuno-detection procedures. Alternatively, several monoclonal antibodies which show good antigen detection quality may be pooled as “synthetic polyclonals” for general detection of the antigen. MAP can also be used to select anti—idiotype antibodies that may structurally resemble the binding pocket on the antigen which the first monoclonal antibody recognizes. For example, mAb1 which is directed against angiopoietin-1 (Ang1) is shown to block Ang1 interaction with its receptor, Tie2. mAb1 is used to immunize inbred mice to generate anti-idiotype antibodies. To determine which clone among the anti-idiotype antibodies generated most likely resembles Ang1's binding site on Tie2 receptor, mAb1 or an Fab fragment (the two domains in an antibody molecule that carry the antigen binding sites) of mAb1 can be linked to a biosensor surface(s) and proteolytically and/or chemically modified as described above. The binding profiles of each anti-idiotype antibody clone as well as Ang1 is collected and analyzed. The response profiles from the anti-idiotype antibody clones that are most similar to that of Ang1 will have the highest probability of resembling Ang1's interacting site with Tie2. An anti-idiotype antibody thus identified may be used instead of Ang1 for certain biological and therapeutic applications.

Yet another application is in discovering and screening for novel chemical modifications on proteins. Among the 20 amino acids that constitute the basic building blocks of all proteins, there are twelve that contain side-chains, which theoretically can be chemically modified. These amino acids are serine, threonine, tyrosine, cysteine, methionine, proline, tryptophan, histidine, lysine, arginine, aspartic acid, and glutamic acid. Traditionally, finding chemical modification conditions that are residue-specific has been difficult. As result, only a few residue-specific amino acid side group chemical modification strategies and reagents are available and widely used to modify protein molecules. Examples of such chemical modification include succinimide chemistry to modify ε-amine on lysine residues; iodination on tyrosine residues; alkylation of cysteine residues; and modifications of carboxylic acid side group of aspartic acid or glutamic acid residues by carbodiimide-mediated chemistry. It has been particularly difficult to find chemical procedures that not only efficiently and specifically modify particular residues but which also maintain the native structure of the protein molecule after the chemical modifications are completed. A set of complete, non-redundant monoclonal antibodies against an antigen molecule identified using MAP will be useful as a reporting system to detect specific structural changes on the antigen surface effected by various chemical modifications. The set of monoclonal antibodies may also be used to find the most desirable chemical modification conditions for the particular antigen. Because all proteins are made of the same 20 amino acids, the reagents or conditions thus identified will be broadly applicable.

MAP may be used to address some basic immunological questions that previously could not be addressed with currently available technologies such as what factors come into play that drive the host (human, mouse, rabbit, etc.) immune system to mount a response which results in the production of antibodies recognizing all possible epitopes on the antigen (immune diversity) on the one hand, versus mounting an immune response which results in the production of antibodies which recognize only a few epitopes (immune dominance) for the same antigen, and how can the host immune response be controlled or modulated such that maximum immune diversity is achieved. These are important questions not only for the development of more and better antibodies for research and drug development, but also for the development of better vaccination formulations against infectious disease and cancer. Traditionally, host immune response diversity toward an antigenic protein could not be systematically studied because there was no efficient way to collect antibody diversity data. The methods described herein provide a promising solution to such problems.

MAP provides an important tool to document the data of epitopic distributions of all positive monoclonal antibodies in each hybridoma experiment simply as a by-product of screening. In addition, the magnitude of epitope diversity coverage may be used to “screen” different immunization conditions and, consequently, questions related to immune diversity of antibody generation by a particular antigen in a particular host can be addressed.

MAP may also be used to study interactions between nucleic acids (DNA, RNA) and proteins. Standard methods routinely used to measure nucleic acid-protein interactions such as gel mobility shift, promoter-reporting assays such as chloramphenicol acetyl transferase (CAT) assay and direct binding assays are generally tedious and time consuming. Here, Applicants propose using a MAP strategy in which DNA (such as, for example, a candidate genomic DNA fragment containing regulatory elements like promoters, enhancers or other regulatory elements) or RNA (such as, for example, precursor RNA or RNA transcripts which are not subject to protein translation but have putative protein interaction functions) are covalently coupled to a biosensor surface followed by individual modification by different endonucleases. Such sets of modified biosensor surfaces can then be used to profile a group of related DNA or RNA binding proteins. The structure-function relationship between nucleic acid sequences and nucleic acid binding proteins may be discovered and verified.

MAP may be used to study carbohydrate-protein interaction studies. Carbohydrate-protein interactions are involved in a wide variety of biological functions including, but not limited to, cellular growth, recognition, adhesion, cancer metastasis, bacterial and viral infections, and inflammation (see Varki (1993) Glycobiology 3:97-101; Lis et al. (1998) Chem. Review 98:637-674). Traditionally, studying carbohydrates and their interactions with proteins has been challenging because carbohydrates (such as oligo- and polysaccharides) not only have complicated structures but it is difficult to determine their primary structures, there is a lack of tools for detecting and analyzing carbohydrate molecules, and many carbohydrate molecules exhibit intrinsic low affinities toward their protein partners (Toone (1994) Curr. Opin. Struct. Biol. 4:719-728). MAP also provides an alternative approach for studying carbohydrate antigens by studying and profiling the epitope distribution of a large group of monoclonal antibodies against their carbohydrate antigen. When a large number of monoclonal antibodies are raised against a carbohydrate antigen and require screening, the carbohydrate antigen molecule may be subjected to similar enzymatic and chemical modification procedures as described in detail above, but substituting proteolytic enzymes with carbohydrate processing enzymes such as exoglycosidases (EndoH, O-Glycosidase and PNGaseF) and endoglycosidases (NANaseI, GALaseI, II, III, IV; HEXase I, II, III, VI; MANase II, etc). Then, hybridomas against the carbohydrate antigen can be profiled into epitope-related siblings by similar procedures and bioinformatic processes. MAP is also useful for studying carbohydrate binding proteins. Proteins that contain carbohydrate-recognition domains (CRDs), such as the calcium-dependant (C-type) lectin family, play crucial roles in biological systems. For example, selectins have crucial roles in leukocyte recruitment in inflammation (Bevilacqua et al. (1993) J. Clin. Invest. 91:79-387) and NKR-P1, a transmembrane member of the C-type lectins, plays a crucial role in activating natural killer (NK) cells and in cytotoxicity (Bezouska et al., Nature (1994) 372:150-157). In addition, carbohydrates from pathogens can be immobilized onto biosensor surfaces and treated with specific carbohydrate processing enzymes, or chemicals that may specifically remove or modify certain monosaccharides within a carbohydrate. Such prepared biosensor surfaces may be used to profile a large group of CRD-proteins into clusters. Based on the nature of each enzyme or chemical treatment, relevant structural information may be revealed.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### General Methods and Materials

Biosensor instruments, biosensor surfaces, and related reagents—The Biacore 3000, 2000, and 1000 instruments are manufactured by Biacore AB Rapsgatan 7 S-754 50 Uppsala, Sweden). Sensor surface chips CM5 or F1 were used for immobilization and modification of the antigen. The 50 mM N-hydroxysuccinimide (NHS) in H₂O; 200 mM N-ethyl-N′-(dimethylaminopropyl) carbodiimide (EDC) in H₂O; and 1M ethanolamine hydrochloride pH 8.5 were prepared using an Amine Coupling Kit purchased from Biacore AB. HBS-EP Buffer: 10 mM Hepes pH 7.4, 150 mM NaCl, 3 mM EDTA, 0.005% surfactant P20. The reagents for Aldehyde coupling were 0.1M sodium cyanoborohydride in 0.1M acetate buffer, pH 4.0; 5 mM hydrazine in H₂O; sodium metaperiodate 50 mM in 100 mM acetate buffer pH 5.5; and 120 mM sodium sulfite in 100 mM acetate buffer, pH 5.5. Carboxymethyl dextran was purchased from Fluka Chemicals (St. Gallen, Switzerland).

Proteolytic enzymes—Modified porcine trypsin, sequencing grade, was purchased from Promega (Madison, Wis., US); Endoproteinase Glu-C, sequencing grade, Endoproteinase Asp-N, sequencing grade, Chymotrypsin, sequencing grade, Endoproteinase Lys-C, sequencing grade, and Endoproteinase Arg-C, sequencing grade, were all purchased from Roche (Roche Diagnostics GmbH, Roche Molecular Biochemicals, Sandhofer Strasse 116, D-68305 Mannheim, Germany).

Chemical reagents—Tris (2-carboxyethyl) phosphine hydrochloride (TCEP•HCl) and N-ethyl-N′-(dimethylaminopropyl) carbodiimide (EDC), N-Hydroxy acetate were obtained from Pierce (3747 N. Meridian Road, P.O. Box 117, Rockford, Ill. 61105, US); Iodoacetamide and Hydrazine were obtained from Sigma (St. Louis, Mo., USA).

Mouse monoclonal antibodies against human Tie2-Fc—Mouse mAbs against human Tie2-Fc were obtained from the following sources: 1) Six previously characterized anti-hTie2 mAbs were either purchased or developed through research collaborations. These antibodies are designated KD5-D10, 33.1/2G10, P15C-B4, 11E11-H11-E7, 11G4-G11, C83711; 2) Forty anti-hTie2 mAbs antibodies were generated and subcloned by conventional hybridoma procedures. These mAbs were stored and used in the form of hybridoma conditioned media. The mAbs are designated F1G1-21, F1G3-7, F4C12-28, F4H5-13, F5A3-30, F10A7-4, F10C12-30, F10G4-10, F11B9-14, F6B9-6, TB2G11-48, M2A6, M3A7, M4A10, M4E2, M4G4, M4H9, M3B9, M3B6, M1A8, K1D4-3, K8H4-11, K8B5-2, K8F4-8, K1F4-5, K4F5-5, K8D4-10, K4F10-6, K5F8-3, K6G6-2, K9F11-10, K1D1-74, K3H3, K5B4-7, K8F7-8, K9B6-19, P5G9-4, K2H4-1, K4F3-5; 3) Sixty-four mAbs against hTie2 were generated by FASTR (FACS-based Autologous Secretary Trap, described briefly in the following paragraphs). These mAbs are designated: 1P2, 2P2, 3P2, 4P2, 5P2, 6P2, 8P2, 9P2, 10P2, 11P2, 12P2, 13P2, 14P2, 15P2,16P2, 17P2, 18P2, 19P2, 20P2, 21P2, 22P2, 23P2, 24P2, 25P2, 26P2, 27P2 28P2, 29P2, 33P2, 35P2, 36P2, 37P2, 38P2, 39P2, 40P2, 41P2, 42P2, 1D3, 1G10, 2P3, 3P3, 4P3, 5P3, 6P3, 7P3, 8P3, 9P3, 10P3, 11P3, 12P3, 13P3, 14P3, 15P3, 16P3, 17P3, 18P3, 19P3, 20P3, 21P3, 22P3, 23P3, 24P3, 25P3, 26P3.

Briefly, the antibodies listed above were prepared as follows: Four Balb/C mice (females) were immunized and boosted with hTie2-Fc fusion protein (250 μg per each mouse) using conventional immunization procedures. Three days after the final boost, the spleen of the best responding mouse was removed and fused with a myeloma cell line engineered to express Fc receptor (SPZ-FcR). About 200 million spleen cells were mixed with approximately 30 million SPZ-FcR cells. 5% of the fusion was plated into four 96-well microtiter plates and the remaining 95% of the fusions were grown in a T75 flask in HAT media for 14 days. Biotinylated human ExTek (His 6-tagged hTie2 ectodomain) was allowed to bind to hybridoma cells expressing anti-hTie2 antibody and followed by addition of avidin-FITC. The top 1% of the bright cell population was collected, a small portion of it was sorted into separate wells for single cell growth. The majority of the top 1% bright cell population pool was divided into two aliquots; one as frozen stock and the other one was put into a T75 flask and allow to grow for another ten days. At the end of the growth period, the total cell population was sorted by the same procedure. This time, 32% of the entire cell population is shifted presumably due to the enrichment from the top 1% from the first FACS sorting. Two clones from this bright cell population were sorted for single cell growth into 96-well plates and the rest of the clones were used in a standard serial dilution cloning procedure. Sixty-five hybridoma clones were collected, among them, 38 clones were from the single cell wells from the first FACS sorting, 25 clones from the single cell well from the second FACS sorting, and 2 clones(1D3,1G10) were from direct single-cell cloning from the second sorting. All the hybridoma conditioned media contained 20% fetal calf serum (FCS) and was diluted prior to MAP experiments with an equal volume of 2X running buffer (20 mM Hepes, pH 7.4, containing 300 mM NaCl and 40 mg/ml carboxymethylated dextran (CMDX)). This method is termed FASTR (FACS-Based-Autologous-Secretary-Trap). For a complete description of the FASTR technology, see WO 02/057423, the contents of which is incorporated herein in its entirety.

The modification of antigen structure is either chemical (which modifies specific amino acid residues in the antigen protein) or enzymatic (which modifies the antigen protein by specifically removing certain sections of polypeptide from the antigen protein). Typically, nine different types of antigen modifications are performed. All modifications are performed within the Biacore instrument, which contains a microfluidity system, a biosensor chip onto which the antigen molecules are immobilized, and a SPR detector. Thus, the modification process can be controlled and monitored in real time.

After all modifications are complete, hybridoma samples are placed in 96-well-microtiter plates and a binding-reporting-regenerating cycle for all of the samples to all of the antigen surfaces is performed by a computer controlled, automated system. Normalized responses are calculated as percentages of antibody binding response to the control (unaltered) antigen surface.

The nine normalized response profiles of the hybridoma samples are then subjected to bioinformatic data analyses. This typically involves further data normalization and application of any or all of the Cluster Algorithms (such as Hierarchical Analysis, Self-Organizing Maps, K-means Method, Principal Component Analysis and Supervised Data Mining) to the normalized data. The results of these analyses will yield a chart, map, or list that outlines the relationships or degrees of similarity of the number of shared characteristics among the tested hybridoma samples. The grouping of the samples can also be achieved by calculating the determinant value of each sample response (surface) matrix, typically for nine-modified surfaces using three-by-three matrix, sorting all samples based on their determinant values, then visually inspecting the original response profiles of each sample to confirm the grouping.

### Example 2

### Preparation of Modified hTie2-Fc on Biosensor Surfaces

hTie2-Fc protein is a 212 kDa dimer containing two 106 kDa hTie2-Fc polypeptides covalently linked by two disulfide bonds provided by the Fc portion of the fusion protein. The protein also contains 10% carbohydrate. hTie2-Fc was coupled to a CM5 biosensor chip surface by a standard NHS/EDC-mediated amine coupling procedure. The amount of hTie2-Fc coupled to each flow-cell surface should be between 3000 to 10,000 RU. To minimize a crowding effect, the preferred coupling density should be around 5000 RU. It is important to couple nearly identical amounts of hTie2-Fc to all four flow-cells so fair comparisons can be made between binding to the three modified flow-cell signals and the non-modified control flow-cell surface.

Six sequencing-grade proteolytic enzymes were used to modify each coupled hTie2-Fc surface: Trypsin, endoproteinase Glu-C and endoproteinase Asp-N to modify flow cell 2, 3, and 4 from the first biosensor chip and chymotrypsin, endoproteinase Lys-C and endoproteinase Arg-C to modify flow cell 2, 3, and 4 from the second biosensor chip. The Biacore 2000 was set to the single flow cell mode at a flow rate of 2 μl/min and 60 μl of 200 μg/ml Trypsin in 0.1M Tris-HCl, pH 8.0 was injected into flow-cell 2. Trypsin digestion could be immediately observed by mass reduction in flow cell 2. The downward curving sensorgram could be observed as a typical proteolytic digestion profile. This indicates that trypsin is specifically removing trypsin-digestible mass. The same dose of enzyme was repetitively injected into the flow-cell until a stable surface was formed. When trypsin digestion was completed on flow-cell 2, 60 μl of 50 μg/ml endoproteinase Glu-C in the same buffer as trypsin was injected into flow-cell 3. Again, the same dose of enzyme was repetitively injected into the same flow-cell until a stable surface was formed. In a similar manner, 60 μl of 50 μg/ml endoproteinase Asp-N in the same buffer was injected into flow-cell 4 to create a stable endoAsp-N modified surface. At the end of the enzyme treatments, the Biacore 2000 was set to all flow-cell mode. A regeneration buffer was run across all the four hTie2-Fc surfaces to generate stable final working surfaces.

75 μl of each hybridoma culture media (containing 20% fetal calf serum) was transferred into a new 96-well microtiter plate and mixed with 75 μl of 2× dilution buffer (20 mM Hepes, pH 7.4, 300 mM NaCl, 0.01% P-20, 40 mg/ml CMDX). The seven pre-characterized monoclonal antibodies against hTie2 were diluted at 10 μg/ml in 1× dilution buffer and placed in 96-well plates. Fresh hybridoma culture medium containing 20% FCS 1:1 diluted with 2× dilution buffer served as a negative control.

Each mAb sample was injected into all four flow-cells, binding signals (RU) from each flow-cell were recorded at the end of the injection and the surfaces were regenerated. The binding/regeneration cycle for each antibody sample was controlled by the Automation Wizard Program provided by the Biacore manufacturer. It took a total of 7 minutes to complete each cycle.

Flow cells 2, 3, and 4 from the second chip containing an identical amount of amine-coupled hTie2-Fc were digested with chymotrypsin, endoproteinase Lys-C, and endoproteinase Arg-C, respectively, in a similar manner as described supra in the preparation of the first chip. The same set of monoclonal antibody samples was injected into all four flow-cells and their binding signals (RU) were collected in the same manner as the first chip.

Chemical modifications. Identical amounts of hTie2-Fc were coupled to all four flow-cells of the third CM5 chip by a standard aldehyde coupling protocol (BIA Applications Handbook, 4.5). The amount of hTie2-Fc coupled to each flow-cell surface should be between 3000 to 10,000 RU, with the preferred coupling amount at around 5000 RU to minimize any crowding effect. To modify the ε-amine of lysine in the hTie2-Fc protein without denaturing its structure, 5 mM sulfo-NHS-acetate dissolved in phosphate buffered saline (PBS) was injected at 5 μl/min into flow-cell 2 for 20 minutes. To modify the carboxylic acid groups of the glutamic acid and aspartic acid residues in the hTie2-Fc protein without denaturing its structure, 200 mM EDC dissolved in H₂O was injected into flow-cell 3 at the same flow rate for 7 minutes followed by an injection of 50 mM hydrazine dissolved in H₂O for 7 minutes. For denaturing treatment of the hTie2-Fc protein, 100 mM TCEP dissolved in 0.1M Tris-HCl, pH 8.0 was injected into flow-cell 4 at the same flow rate for 20 minutes followed by injection of 100 mM iodoacetamide dissolved in 0.1M Tris-HCl, pH 8.0. At the end of the treatments, the Biacore 2000 was set to all flow-cell mode. A regeneration buffer was injected into all four hTie2-Fc surfaces three times to generate a stable final working surfaces.

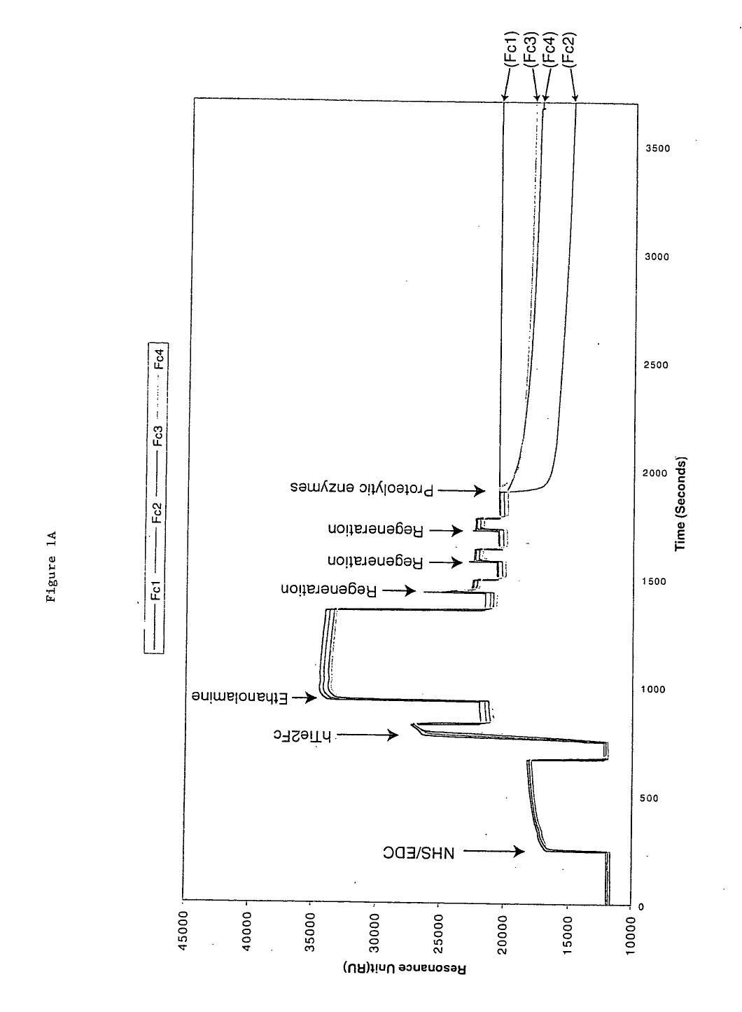

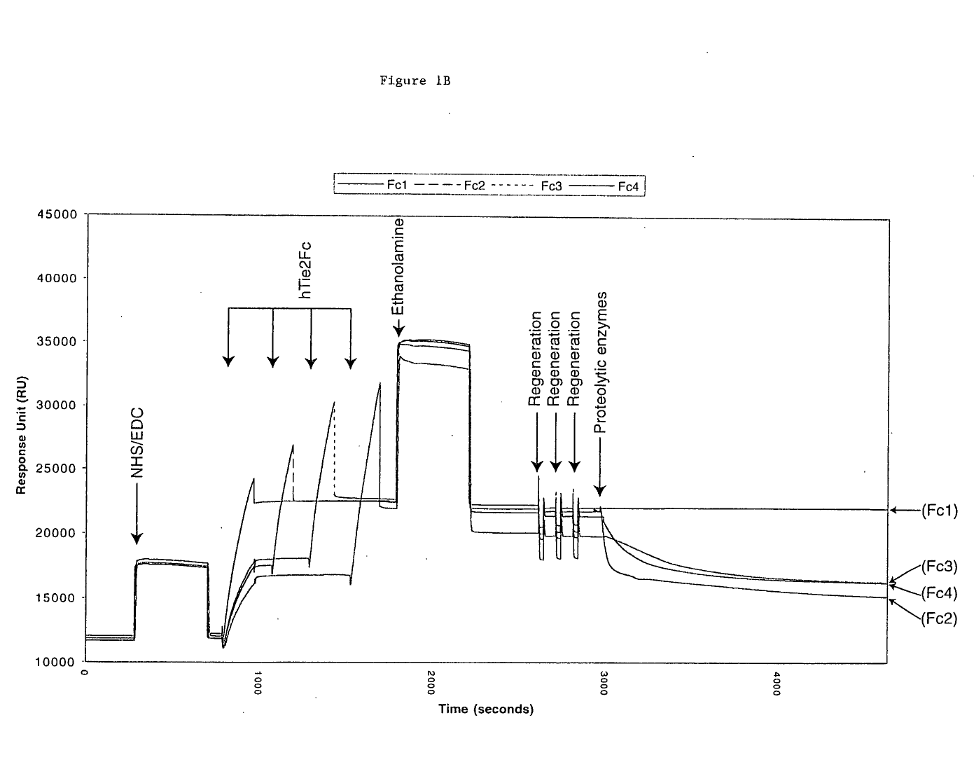

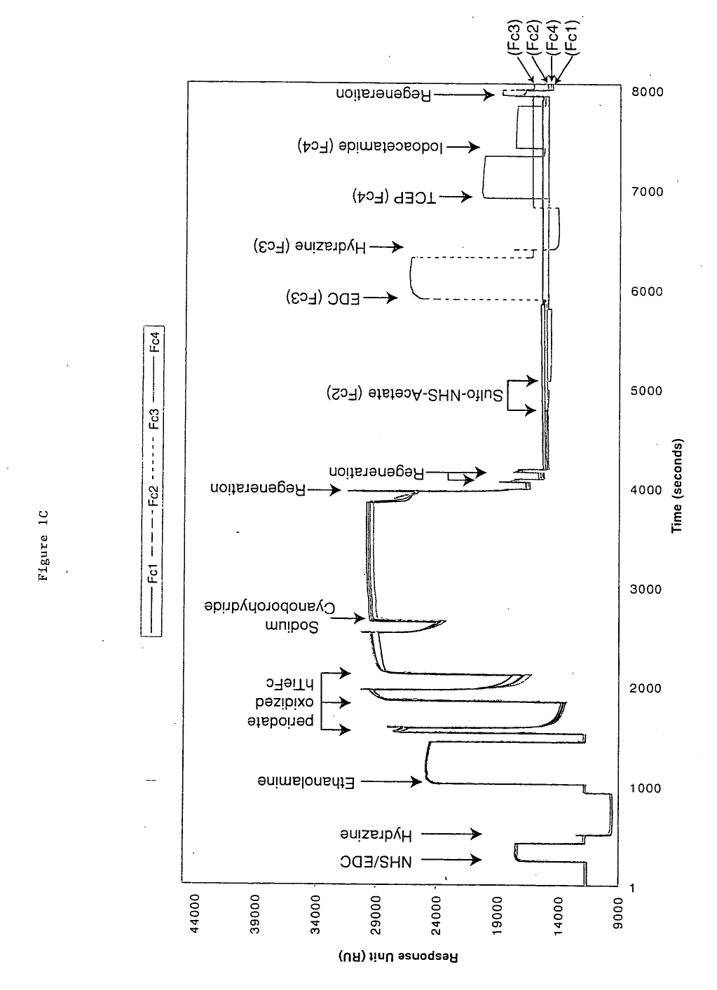

The binding of each anti hTie2-Fc antibody to the third chip containing chemically modified aldehyde-coupled hTie2-Fc was performed in the same way as the other two chips. FIG. 1A-1C are representative Biacore® sensorgrams of modified antigen surfaces. FIG. 1A shows a Biacore® Sensorgram of a control human Tie2-Fc (hTie2-Fc) biosensor surface and three proteolytically modified hTie2-Fc biosensor surfaces which were generated by digestion with trypsin, endoproteinase Glu-C, or endoproteinase Asp-N, respectively. FIG. 1B shows a Biacore® Sensorgram of a control hTie2-Fc sensor surface and three proteolytically modified human Tie2-Fc sensor surfaces were generated by digestion with chymotrypsin, endoproteinase Lys-C, or endoproteinase Arg-C, respectively. FIG. 1C shows a Biacore® Sensorgram of a control hTie2-Fc sensor surface and three chemically modified hTie2-Fc sensor surfaces were generated by chemical treatments with Sulfo-NHS-Acetate, EDC/Hydrazine, or TCEP/Iodoacetamide, respectively.

### Example 3

### Generating Monoclonal Antibody Binding Profiles Using the Biacore 2000

Hybridoma conditioned media samples were diluted at 1:1 ratio with 2× dilution buffer (20 mM Hepes, pH 7.4, 300 mM NaCl, 6 mM EDTA, 0.01% Surfactant P20 and 40 mg/ml CMDX) in 96-well microtiter plates. The binding of each monoclonal hybridoma sample to each biosensor chip that contained one unmodified hTie2-Fc surface and three separately modified hTie2-Fc surfaces was performed automatically under the control of Biacore software.

When all of the mAb binding data to the three separate chips which contain the nine modified hTie2-Fc surfaces and three unmodified hTie2-Fc control surfaces were collected, all of the nine response RU values of each antibody to the nine modified hTie2-Fc surfaces were converted into response ratios to that of the unmodified controls.

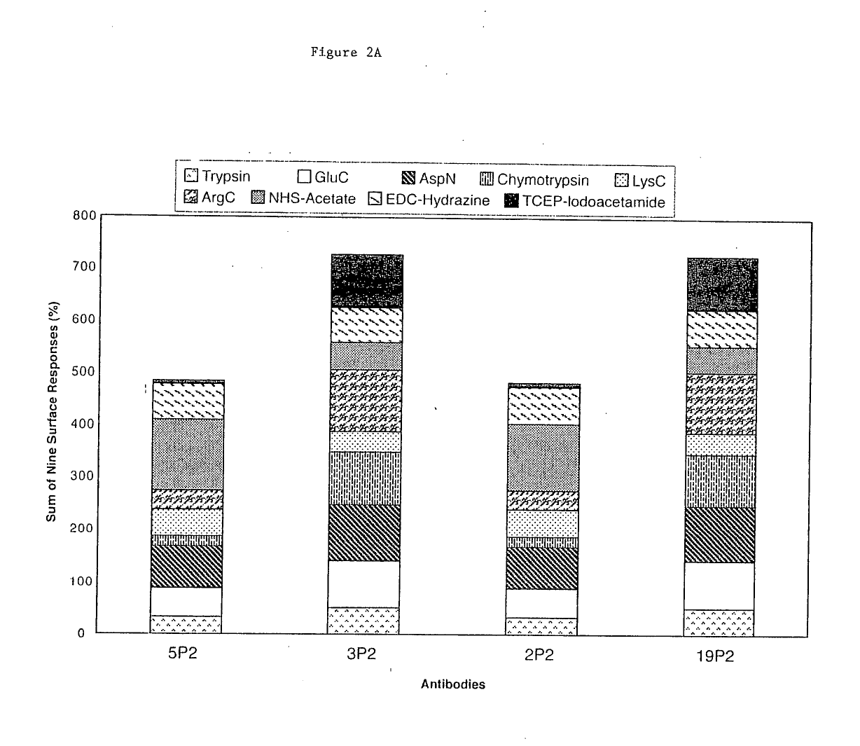

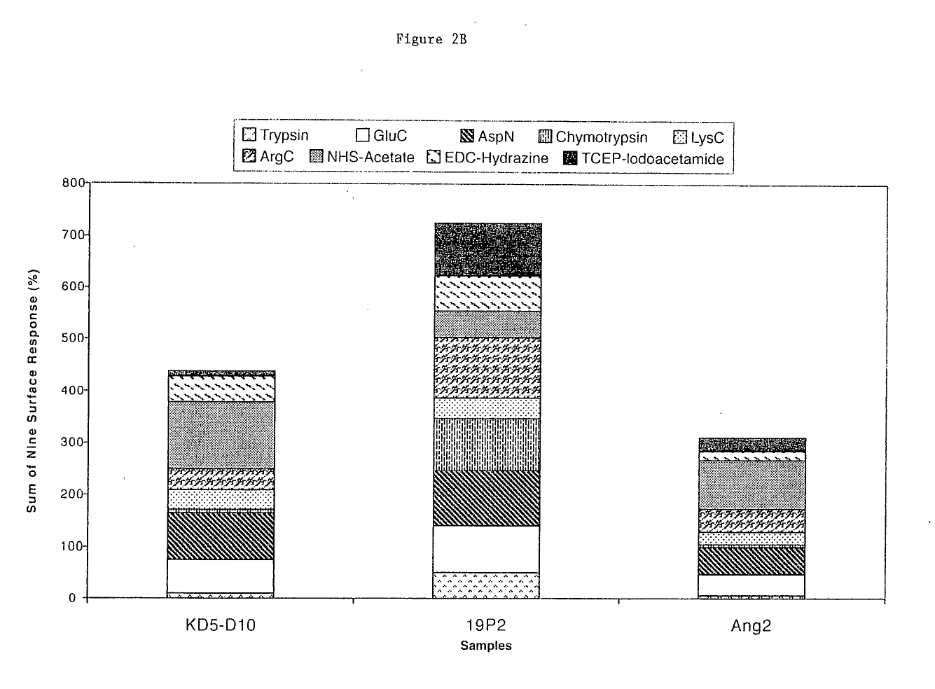

The response data of all the tested anti-hTie2 mAbs (110 mAbs and 174 primary hybridoma conditioned media supernatants and 6 hTie2 ligands) preparations were subjected to bioinformatic data analyses as described above. The results of these mAbs epitope cluster distributions are shown by typical pattern recognition (non supervised) display methods. One of such display methods are hierarchical trees (Dendrograms) which outline the cluster relationships of the monoclonal antibodies in a tree-like arrangement. In the hierarchical tree, antibodies that are likely share epitopes will be linked together by relatively shorter “arms”, where those that unlikely share epitopes will be linked by relatively longer “arms”. The response data of all of the tested anti-hTie2 mAbs can also be expressed as gradated color bars that indicates the nine normalized responses of each antibody. Antibodies can then be clustered into individual groups based on their color bar profiles. In addition, the response data of all of the tested anti-hTie2 monoclonal antibodies can be subjected to matrix-determinant calculations. The determinant value derived from a particular matrix is a single number that uniquely defines that antibody matrix (it is the vector (orientation) of that antibody in the nine dimensional data space) All samples can then be sorted based on their determinant values, followed by visually inspecting the original response profiles of each sample to confirm the grouping. For examples, FIG. 2A shows the response profiles of four anti-human Tie2 (anti-hTie2) mAbs. Small amounts of conditioned media containing mAbs from 4 different hybridoma cultures were injected over three sensor chips, each chip containing control and three modified hTie2-Fc biosensor surfaces as described in FIGS. 1A-1C. The binding signal from each modified biosensor surface was converted into percentage of control (non-modified hTie2-Fc) biosensor surface within the same chip. The gradated bar represents a profile of response percentages of all nine modified hTie2-Fc biosensor surfaces with each of the anti-hTie2 mAb. Four such exemplary profiles are shown. FIG. 2B shows a comparison of the response profiles of two anti-hTie2-Fc mAbs with the response profile of human angiopoietin-2 (Ang2), a natural ligand of hTie2. Small amounts of conditioned media containing mAbs from 2 different hybridoma cultures were injected over three sensor chips, each chip containing control and three modified hTie2-Fc biosensor surfaces as described in FIGS. 1A-1C. The binding signal from each modified hTie2-Fc surface was converted into percentage of control (non-modified hTie2-Fc) biosensor surface within the same chip. The gradated bar graph represents a profile of response percentages of all 9 modified hTie2-Fc biosensor surfaces with either 2 anti-human Tie2 mAbs or Ang2.

### Example 4

### Verification of Antibody Clusters by Biacore Epitope Mapping

Monoclonal antibodies from two different functional groups (or clusters or bins) as determined MAP can be verified by other methods such as ELISA, competition assay, etc. In this example, a Biacore epitope mapping assay typically performed by Biacore 1000 was used. Antibodies from two different functional groups should not interact with the same epitope. Therefore, the binding of a first antibody from one cluster to the immobilized antigen should not preclude binding of a second antibody from a different cluster to any significant extent. Conversely, antibodies from the same cluster should exhibit near complete competition with each other when binding to their antigen.

hTie2-Fc was coupled to CM5 by amine coupling at a density of about 1000 RU. The first antibody sample was injected into this hTie2 surface to reach saturation binding, followed by injection of a second antibody sample. This process was repeated such that the first antibody was always injected at saturation levels and then followed by injection of a different antibody to determine whether the binding of the first antibody could prevent the binding of each of the rest of the tested antibodies to the human Tie2-Fc surface.

10 randomly chosen mAbs from cluster C30 (total 30 members) to a hTie2 surface. Each mAb was bound to 1500RU of the amine-coupled hTie2 surface at a near-saturable level followed by a second antibody binding of the same mAb or each of the other nine different mAbs. The result showed that all of the ten clones inhibit each other binding to the hTie2 antigen (Fig3A.)

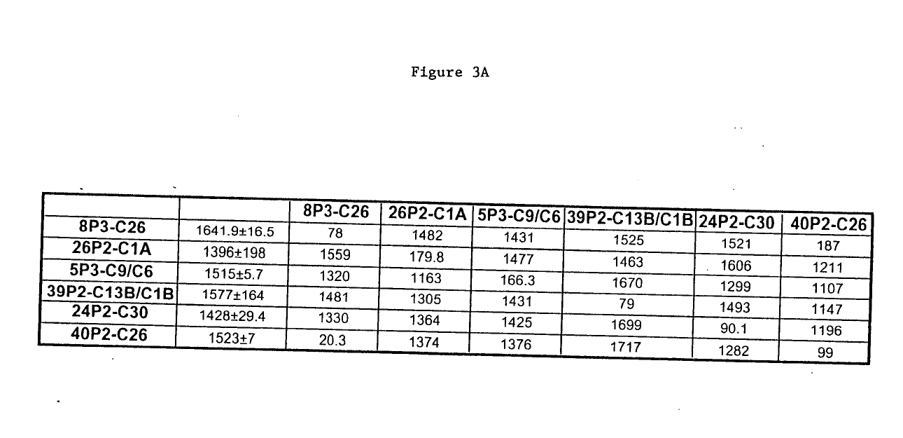

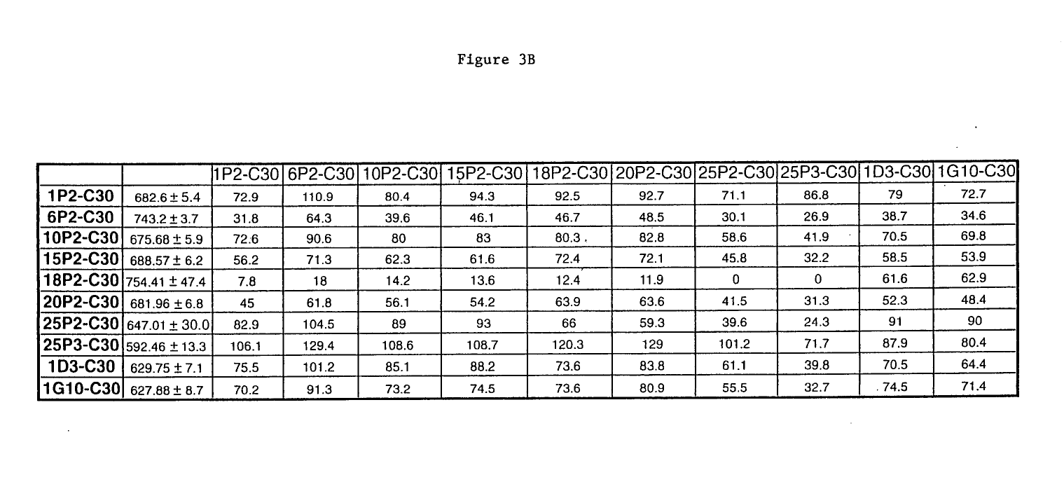

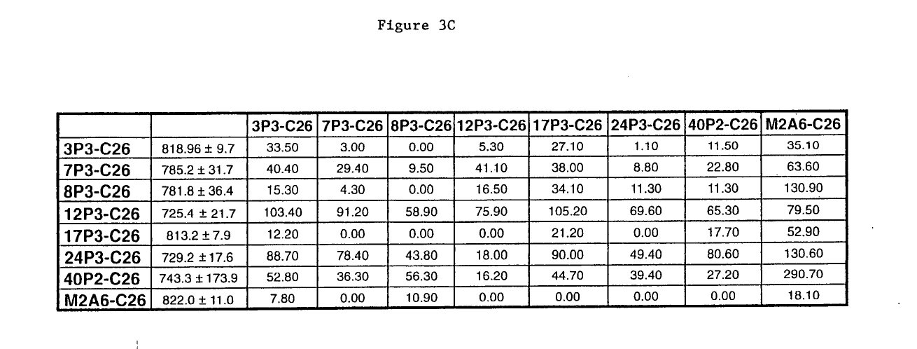

Six antibodies were chosen from another cluster C9/C6 determined by MAP to represent six members within a cluster. These antibodies are designated 2P2, 5P2, 2P3, 5P3, 9P3, and 20P3. The result showed that the clones inhibit each other bindings to the hTie2 antigen (FIG. 3B). Six mAbs were chosen from five separate functional groups determined by MAP. These mAbs are designated 26P2, 39P2, 2P2(C9/C6), 8P3(C26), 24P2(C30), and 40P2(C26). that the 5 mAbs from different clusters determined by MAP did not inhibit each other binding to the hTie2 antigen while clone 40P2 and 8P3 that from the same cluster determined by MAP did inhibit each other binding to the Tie2 antigen, even though the two clones do not belong to the same genetic sibling.

### Example 5

### Epitope Mapping Using Human Tie2-derived Peptide Dot-blot

Monoclonal antibody functional groups identified using MAP may also be verified using a hTie2 primary sequence-derived peptide array. Peptides derived from the human Tie2 extracellular domain or overlapping peptides to cover the entire Tie2 extracellular domain are prepared as dot arrays on a PVDF membrane. Antibodies representing different functional groups or antibodies from the same functional group are incubated with the PVDF membranes containing the peptide arrays followed by a standard dot blotting and staining. Antibodies from the same functional group, which recognize the same epitope, should display identical binding patterns on the peptide array sheet. Conversely, antibodies from different functional groups, which recognize a different epitope on the hTie2 antigen, should display a different binding pattern to the peptide array.

### Example 6

### Confirming Genetic Functional Groups by Directly Sequencing Each Antibody Gene

Approximately 10,000 cells from each hybridoma clone were used to isolate total RNA followed by RT-PCR. RT-PCR was performed using a kit from Qiagen (Cat# 210212). The primer pair capable of detecting the murine immunoglobulin heavy chain (IgG1) variable region is a mixture of 7 degenerate 5′ primers and a single non-degenerate IgG1 3′ primer (Wang et al. (2000) J. Immunol. Methods 233:167-177). These 5′ primers were designated MH1, MH2, MH3, MH4, MH5, MH6, and MH7. The 3′ primer was designated IgG1. The PCR product obtained from each hybridoma clone was subsequently sequenced and the nucleotide sequences of each hybridoma heavy chain PCR product were verified and compared. The results show that the 61 FASTR-generated anti-hTie2 monoclonal antibodies have six unique heavy chain sequences. Antibodies within the same functional group share identical heavy chain sequences with one exception, clone 40P2(C26), which shares a nearly identical MAP binding profile in with the C26 group, but has a unique heavy chain nucleotide sequence. These results suggest 1) the four clusters (C30, C9/C6, C1A, C13B/C1B) are genetic siblings and 2) cluster C26 contains 21 genetically identical clones and one functional sibling (40P2). These genetic sibling clusters are: C30: 1P2, 4P2, 6P2, 9P2, 10P2, 11P2, 12P2, 13P2, 14P2, 15P2, 16P2, 17P2, 18P2, 20P2, 22P2, 23P2, 24P2, 25P2, 27P2, 33P2, 35P2, 36P2, 37P2, 38P2, 41P3, 42P2, 1D3, 1G10, 25P3, 26P3; C26: 3P3, 4P3, 6P3, 7P3, 8P3, 10P3, 11P3, 12P3, 13P3, 14P3, 15P3, 16P3, 17P3, 18P3, 19P3, 21P3, 22P3, 23P3, 24P3, 21P2, 28P2, 29P2, 33P2; C9/C6: 2P2, 5P2, 2P3, 5P3, 9P3, 20P3; C1A: 26P2; C13B/C1B: 39P2; and C26: 40P2.

Biacore epitope mapping data above confirmed that clone 40P2 does compete with clone 8P3(C26).

### Example 7

### Antibody K1D4 Mimicking Clone 39P2 Stimulated hTie2 Receptor Phosphorylation

All FASTR-generated hTie2 monoclonal antibodies have been tested for their ability to stimulate hTie2 receptor phosphorylation in EAHy926 cells. The experiments were conducted with T-75 flasks of confluent EA cells starved for 2 hrs in DMEM High Glucose. Each cell flask is challenged with a protein of interest in 1.5 ml/flask DMEM High with 0.1% BSA for different time periods. The cell from each flask is lysed with 1.5 ml of RIPA buffer (Tris 20 mM pH 7.5, NaCl 150 mM, NaF 50 mM, Na Vanadate 1 mM, benzamidine 5 mM, EDTA 1 mM, NP40 1%, Na Deoxycholate 0.5%, SDS 0.1%, Leupeptin/Aprotinin 10 μg/ml, PMSF 1 mM). The supernatant from each lysed cell sample is immunoprecipitated (IP) by a rabbit polyclonal anti hTie2 antibodies(RG133, 5 μg/ml), biotinylated antirabbit Antibody (5 μg/ml) and NeutrAvidin beads (Pierce). The IP products are separated by SDS PAGE and blotted onto PVDF. The phosphorylation signals are detected with 4G10 anti-Phosphotyrosine Ab (Upstate Biotech) and HRP-conjugated secondary antibody and then developed by ECL (Amersham). Among all tested FASTR clones, 39P2 exhibited the strongest ability to stimulate hTie2 phosphorylation. MAP results predict clone K1D4-3 among 40 hTie2 monoclonal antibodies generated by conventional hybridoma procedure will exhibit similar potency as that of 39P2 in stimulating hTie2 receptor phosphorylation based on the MAP profile of 39P2 and K1D4-3 (39P2 and K1D4-3 are grouped as one functional cluster). The results from a similar phosphorylation experiment as described above show that only K1D4-3, not the other 39 hTie2 monoclonal antibodies generated by conventional hybridoma procedure, exhibits a potent ability to stimulate hTie2 receptor phosphorylation. All FASTR-generated hTie2 mAbs have been tested for their ability to stimulate hTie2 receptor phosphorylation in EAHy926 cells. The experiments were conducted as the following: Aliquots of EAHy926 cells were cultured in 10 cm dishes to near confluence. The cells were washed twice with PBS, and each antibody sample diluted with DMEM to 0.5 μg/ml was added to each 10 cm dish. The cell were then incubated at 37° C. for various time from 30 min. to 2 hrs. At the end of incubation, each dish was washed three times with PBS, and the cells were collected. Each cell pellet was dissolved in PBS containing 0.5% Chaps, a protease-inhibitor cocktail, and 5 mM Vanadate. The solubilized hTie2 receptors from each sample were recovered by immunoprecipitation (IP) with anti-hTie2 mAb clone 33.1. The IP products were run on SDS-PAGE followed by Western Blotting with the anti-phosphotyrosine mAb 4G10 coupled to HRP-goat-anti-mouse IgG detection. Among all tested FASTR clones, 39P2 exhibited the strongest ability to stimulate hTie2 phosphorylation. In a similar assay, the MAP-identified clone K1D4, which has very similar MAP profile as 39P2 (39P2 and K1D4 are grouped as one functional cluster) was tested for its ability to stimulate hTie2 phosphorylation. In this experiment, K1D4 and 5 clones generated against hTie2 using conventional hybridoma procedures were tested for their ability to stimulate hTie2 phosphorylation. The results show that only K1D4 exhibited a potent ability to stimulate hTie2 receptor phosphorylation.

### Example 8

### MAP Analyses of Two Antibody-antigen Systems

MAP analyses have been applied to 25 mAbs raised against human recombinant IL-6 (hIL-6) protein, in which hIL-6 was coupled to all three CM5 chips by amine-coupling procedure followed by the same enzymatic and chemical procedures to each corresponding flow-cell as described above. The 25 monoclonal antibodies were clustered into 4 epitope groups and the result was confirmed by a conventional pair-wise competition assay as described above.

MAP analyses have been applied to 79 mAbs raised against IL-4/13 trap, a chimeric fusion protein comprising the human IL-4 receptor α-domain, the human IL-13 receptor α-domain, and a human IgG1 Fc. In this analysis, IL-4/13 trap was amine-coupled to chip 1 and 2 and aldehyde-coupled to chip 3, followed by the same enzymatic and chemical procedures as described in above. The MAP profile was able to sort the 79 monoclonal antibodies into three main groups: antibodies directed to the IL-4 receptor a domain, antibodies directed to the IL-13 receptor a domain, and antibodies directed to the IgG1 Fc domain. MAP procedure further clustered 26 of the IL-13 receptor α domain mAbs into 6 epitope groups within IL-13 receptor α domain, 48 IL-4 receptor α domain mAbs into 5 epitope groups within IL-4 receptor α domain, and 5 IgG1 Fc domain mAbs into 3 epitope groups within IgG1 Fc domain.

Although the foregoing invention has been described in some detail by way of illustration and examples, it will be readily apparent to those of ordinary skill in the art that certain changes and modifications may be made to the teachings of the invention without departing from the spirit or scope of the appended claims.

We claim,

1. A method of identifying a site of interaction between a first and second macromolecule, comprising:
(a) immobilizing the first macromolecule onto at least two biosensor surfaces;
(b) treating each biosensor surface containing the immobilized first macromolecule with a different agent capable of altering the structure of the immobilized first macromolecule;
(c) exposing each treated biosensor surface to the second macromolecule;
(d) determining an interaction profile of the second macromolecule to the immobilized and treated first macromolecule; and
(e) identifying a site of interaction between the first and second macromolecules based on the interaction profile.

2. The method of claim 1, wherein the agent capable of altering the structure of the first macromolecule is an enzyme.

3. The method of claim 2, wherein the enzyme is a proteolytic enzyme.

4. The method of claim 3, wherein the proteolytic enzyme is selected from the group consisting of trypsin, endoproteinase Glu-C, endoproteinase Asp-N, chymotrypsin, endoproteinase Lys-C, and endoproteinase Arg-C.

5. The method of claim 2, wherein the enzyme is selected from the group consisting of a lipase, amylase, and endonuclease.

6. The method of claim 1, wherein the agent capable of altering the structure of the first macromolecule is a chemical agent.

7. The method of claim 6, wherein the chemical agent is selected from the group consisting of Tris (2-carboxyethyl) phosphine hydrochloride (TCEP•HCl), N-ethyl-N′-(dimethylaminopropyl) carbodiimide (EDC), iodoacetamide, hydrazine, p-hydroxyphenylglyoxal (HPG), hydrogen peroxide, N-bromosuccinimide, N-acetylimidazole, tetranitromethane, arsanilic acid, dansyl chloride, glutaraldehyde, ninhydrin, and diethylpyrocarbonate (DEPC).

8. The method of claim 6, wherein the first macromolecule is a lipid and the chemical agent is selected from the group consisting of reactive compounds that modify lipids by N-ethyl-N′-(dimethylaminopropyl) carbodiimide (EDC)-mediated chemistry.

9. The method of claim 6, wherein the first macromolecule is a carbohydrate and the chemical agent is selected from the group consisting of primary amine-containing compounds that modify carbohydrates by periodate-mediated chemistry.

10. The method of claim 6, wherein the first macromolecule is a nucleic acid and the chemical agent is a methylating agent.

11. The method of claim 1, wherein the biosensor surface is a Biacore biosensor surface.

12. The method of claim 1, wherein the biosensor surface is an IAsys® biosensor surface, a SPR670 biosensor surface, a Bio-Suplar II biosensor surface, or a Spreeta™ biosensor surface.

13. The method of claim 1, wherein the first macromolecule and the second macromolecule are selected from the group consisting of:
(a) proteins, wherein the proteins are different proteins;
(b) a protein and a carbohydrate;
(c) a protein and a ligand;
(d) a protein and a nucleic acid; and
(e) a ligand and a receptor.

14. The method of claim 13, wherein the ligand is selected from the group consisting of a carbohydrate, nucleic acid, small molecule, peptide, and lipid.

15. The method of claim 14, wherein the nucleic acid is DNA or RNA.

16. The method of claim 13, wherein the protein is a transcription factor.

17. A method of sorting antigen-specific monoclonal antibodies (mAbs) into functional groups, comprising:
(a) immobilizing the antigen onto at least two biosensor surfaces;
(b) treating each biosensor surface with a different agent, wherein each agent is capable of altering the structure of the immobilized antigen;
(c) exposing each treated biosensor surface to the antigen-specific mAbs;
(d) determining the binding profile of the monoclonal antibodies to each treated biosensor surface; and
(e) sorting the mAbs into functional groups based on a binding profile of the monoclonal antibodies to each treated biosensor surface, wherein mAbs that exhibit similar binding profiles to each treated sensor surface are sorted into the same functional group.

18. The method of claim 17, wherein the agents capable of altering the structure of the immobilized antigen are enzymes.

19. The method of claim 18, wherein the enzymes are proteolytic enzymes selected from the group consisting of trypsin, endoproteinase Glu-C, endoproteinase Asp-N, chymotrypsin, endoproteinase Lys-C, and endoproteinase Arg-C.

20. The method of claim 17, wherein the agents capable of altering the structure of the immobilized antigen are chemical agents selected from the group consisting of Tris (2-carboxyethyl) phosphine hydrochloride (TCEP•HCl), N-ethyl-N′-(dimethylaminopropyl) carbodiimide (EDC), iodoacetamide, hydrazine, p-hydroxyphenylglyoxal (HPG), hydrogen peroxide, N-bromosuccinimide, N-acetylimidazole, tetranitromethane, arsanilic acid, dansyl chloride, glutaraldehyde, ninhydrin, and diethylpyrocarbonate (DEPC).

21. The method of claim 17, wherein the biosensor surface is a Biacore sensor surface an IAsys® biosensor surface, a SPR670 biosensor surface, a Bio-Suplar II biosensor surface, or a Spreeta™ biosensor surface.
